(19) United States
(12) Patent Application Publication
(10) Pub. No.: US 2020/0157599 A9
(43) Pub. Date: May 21, 2020

(54) NEGATIVE-POSITIVE ENRICHMENT FOR NUCLEIC ACID DETECTION

(71) Applicant: GENETICS RESEARCH, LLC, D/B/A ZS GENETICS, INC., Wakefield, MA (US)

(72) Inventor: Anthony P. Shuber, Wakefield, MA (US)

(21) Appl. No.: 16/007,427

(22) Filed: Jun. 13, 2018

Related U.S. Application Data
(60) Provisional application No. 62/656,592, filed on Apr. 12, 2018.
(60) Provisional application No. 62/568,121, filed on Oct. 4, 2017.
(60) Provisional application No. 62/526,091, filed on Jun. 28, 2017.
(60) Provisional application No. 62/519,051, filed on Jun. 13, 2017.

(51) Int. Cl. C12Q 1/6806; C12N 9/22; C12N 15/11

(52) U.S. Cl. CPC C12Q 1/6806; C12N 9/22; C12N 2800/80; C12N 2310/20; C12N 15/11

(57) ABSTRACT

The invention provides methods of detecting a feature of interest in a nucleic acid sample by negatively and positively enriching the sample for segments that contain the feature of interest. Negative enrichment may include digestion of nucleic acids that do not contain the segments, and positive enrichment may include purification of the segments. The methods are useful for diagnostic of genetic elements, e.g., elements indicative of cancer.

### CROSS-REFERENCE TO RELATED APPLICATIONS

This application claims the benefit of, and priority to, U.S. Provisional Patent Application No. 62/656,592, filed Apr. 12, 2018, U.S. Non-provisional patent application Ser. No. 15/877,619, filed Jan. 23, 2018, U.S. Provisional Patent Application No. 62/568,121, filed Oct. 4, 2017, U.S. Provisional Application No. 62/526,091, filed Jun. 28, 2017, and U.S. Provisional Application No. 62/519,051, filed Jun. 13, 2017, the contents of each of which are incorporated by reference.

### FIELD OF THE INVENTION

The disclosure relates to molecular genetics.

### BACKGROUND

Each year over 1.5 million people are newly diagnosed with cancer in the United States alone. Cancer kills over 500,000 Americans annually, incapacitates many more, and disrupts the lives of families and friends of those afflicted. Cancer exacts an economic toll as well: the estimated direct medical costs for cancer treatment in the United States in 2014 were $87.8 billion, and some sources project that this number could exceed $200 billion by 2020.

One reason that cancer is so costly in both human and financial terms is that existing methods of detecting cancer are inadequate. Early and accurate diagnoses are critical to effective treatment of cancer. However, many cancers present with nonspecific clinical symptoms, and diagnosis only occurs when the disease has reached a stage at which it cannot be successfully treated. In addition, most diagnostic methods fail to identify the cause of the cancer and thus provide little guidance on how to treat it. Moreover, due to the lack of sensitivity of detection methods, progression of the disease and its response to therapeutic intervention are difficult to monitor. Consequently, physicians lack the tools to make timely, informed decisions on therapeutic intervention, and cancer continues to kill millions of people each year.

### SUMMARY

The invention provides methods that include both negative and positive enrichment of an element, such as a mutation indicative of cancer, in a nucleic acid sample. The negative enrichment includes protecting a nucleic acid of interest and digesting unprotected nucleic acids. The nucleic acid that was protected is then purified from the sample in the positive enrichment. The two-step process results in much greater enrichment of the segment in sample than can be achieved by negative or positive enrichment alone. Consequently, the methods of the invention allow detection of elements present at low quantities in nucleic acid samples.

Because most cancers arise from acquired mutations resulting from environmental insults, the methods of the invention are useful for diagnosing cancer. In particular, the ability to detect mutations or other elements present at low quantities permits diagnosis of cancer at early stages when effective treatment is possible. Detection of specific mutations that cause cancer provides insight into the mechanistic basis of the disease. Consequently, the methods also allow clinicians to provide prognoses for patients and predict the efficacy of a particular course of treatment. The sensitivity of the methods also makes them useful for determining the stage of cancer and monitoring disease progression.

The methods of the invention are also useful for other diagnostic applications that require detection of low-abundance nucleic acids. For example, low levels of fetal DNA are present in the blood of pregnant females. Thus, the methods allow diagnosis of genetic abnormalities in unborn children.

In certain aspects, the invention provides a method for detecting nucleic acid in a sample. The method includes protecting a nucleic acid of interest in a sample by binding proteins to ends of the nucleic acid, digesting unprotected nucleic acid, enriching the sample for the nucleic acid, and detecting the nucleic acid. In certain embodiments, the proteins each comprise a Cas endonuclease complexed with a guide RNA that targets the Cas endonuclease to an end of the nucleic acid. Catalytically inactive Cas proteins (dCas) may be used. Digesting the unprotected nucleic acid may include introducing an exonuclease into the sample. In some embodiments, the enriching step comprises connecting the protected nucleic acid to a particle (such as an agent that binds to one or both of the proteins) or column and removing other components of the sample. The particle may be a magnetic particle, and the enriching step may include applying a magnetic field to separate the particle-bound protected nucleic acid from the other components. The enriching step may include applying the sample to a column, e.g., an HPLC column. Optionally, the protected segment is separated from unprotected nucleic acid by size exclusion, ion exchange, or adsorption. Preferably the digesting step comprises exposing the unprotected nucleic acid to one or more exonucleases.

The detecting step may include DNA staining, spectrophotometry, sequencing, fluorescent probe hybridization, fluorescence resonance energy transfer, optical microscopy, and electron microscopy. Detecting the nucleic acid may include identifying a mutation in the nucleic acid. Identifying the mutation may include sequencing the nucleic acid (e.g., on a next-generation sequencing instrument), allele-specific amplification, and hybridizing a probe the nucleic acid.

The sample may include blood or plasma, and the nucleic acid may be DNA from a tumor. The sample may be a liquid biopsy sample, such that the nucleic acid comprises circulating tumor DNA. In some embodiments, the sample comprises maternal plasma, and wherein the nucleic acid comprises fetal DNA.

In an aspect, the invention provides methods for detecting a feature of interest in a nucleic acid sample. The methods include protecting a segment that includes the feature of interest by binding proteins to ends of the segment, digesting unprotected nucleic acid, enriching the sample for the segment, and detecting the segment. The feature of interest may be at or near an end of the segment, and one of the proteins may bind to the feature of interest.

Any suitable method may be used to enrich the sample for the segment, i.e., for the positive enrichment. The positive enrichment may include separating the protected segment from some or all of the unprotected nucleic acid. The positive enrichment may include binding protected segment to a particle. The particle may include magnetic or paramagnetic material. The positive enrichment may include applying a magnetic field to the sample. The particle may include an agent that binds to a protein bound to an end of the segment. The agent may an antibody or fragment thereof. The positive enrichment may include chromatography. The positive enrichment may include applying the sample to a column. The positive enrichment may include separating the protected segment from some or all of the unprotected nucleic acid by size exclusion, ion exchange, or adsorption. The positive enrichment may include gel electrophoresis.

Each of the proteins may independently be any protein that binds a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). The protein may be a catalytically inactive form of a nuclease, such as a programmable nuclease described above. The protein may be a transcription activator-like effector (TALE). The protein may be complexed with a nucleic acid that guides the protein to an end of the segment. For example, the protein may be a Cas endonuclease in a complex with one or more guide RNAs.

The unprotected nucleic acid may be digested by any suitable means. Preferably, the unprotected nucleic acid is digested by one or more exonucleases.

The segment may be detected by any means known in the art. For example and without limitation, the segment may be detected by DNA staining, spectrophotometry, sequencing, fluorescent probe hybridization, fluorescence resonance energy transfer, optical microscopy, or electron microscopy.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The feature of interest may be any feature of a nucleic acid. The feature may be a mutation. For example and without limitation, the feature may be an insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism.

The segment may be from a sub-population of nucleic acid within the nucleic acid sample. For example, the segment may contain cell-free DNA, such as cell-free fetal DNA or circulating tumor DNA.

The nucleic acid sample may be from any source of nucleic acid. The sample may be a liquid or body fluid from a subject, such as urine, blood, plasma, serum, sweat, saliva, semen, feces, or phlegm. The sample may be a liquid biopsy.

In an aspect, the invention provides methods for detecting a mutation in a nucleic acid sample. The methods include protecting a segment that includes a mutation by binding a protein to the mutation and another protein to the segment, digesting unprotected nucleic acid, enriching the sample for the segment, and detecting the segment. The method may include any of the elements described above.

Embodiments of the invention use proteins that are originally encoded by genes that are associated with clustered regularly interspaced short palindromic repeats (CRISPR) in bacterial genomes. Preferred embodiments use a CRISPR-associated (Cas) endonuclease. For such embodiments, the binding protein in a Cas endonuclease complexed with a guide RNA that targets the Cas endonuclease to a specific sequence. The complexes bind to the specific sequences in the nucleic acid segment by virtue of the targeting portion of the guide RNAs. When the Cas endonuclease/guide RNA complex binds to a nucleic acid segment, the complex protects that segment from digestion by exonuclease. When two Cas endonuclease/guide RNA complexes bind to a segment, they protect both ends of the segment, and exonuclease can be used to promiscuously digest un-protected nucleic acid leaving behind an isolated fragment—the segment of DNA between two bound complexes.

Structural alterations are detected using guide RNAs designed to hybridize to targets flanking a boundary of the alteration. Using two such guide RNAs, first and second Cas endonucleases will bind to the nucleic acid in positions that flank the breakpoint, thereby defining and protecting the segment of nucleic acid that includes the breakpoint. In the absence of the alteration, the two Cas endonuclease/guide RNA complexes will not bind to the same strand, and all of the nucleic acid will end up digested upon exposure to exonuclease. Small mutations, such as substitutions or small indels, are detected using an allele-specific guide RNA—a guide RNA that binds the Cas endonuclease exclusively to the mutation of interest. An allele-specific guide RNA may be used in conjunction with another guide RNA that binds a Cas endonuclease to the same nuclei acid, so that the two Cas endonuclease/guide RNA complexes define and protect a segment between them, but only do so when the small mutation is present in the sample. Accordingly, the invention provides methods for selectively isolating segments of nucleic acid that contain clinically relevant mutations.

Protecting a segment of target nucleic acid with two binding proteins while promiscuously digesting unprotected nucleic acid may be described as a negative enrichment for the target. Embodiments of negative enrichment may be used for the detection of “rare events” where a specific sequence of interest makes up a very small percentage of the total quantity of starting material. Specifically, negative enrichment techniques may be used to detect specific mutations in circulating tumor DNA (ctDNA) in the plasma of cancer patients, or specific mutations of interest potentially associated with fetal DNA circulating in maternal plasma. In addition negative enrichment analysis can be applied to purified circulating tumor cells (CTCs).

In one embodiment a single or a cocktail of Cas9/gRNA complex(s) are created with the gRNA(s) designed specifically to target a region in the genome known to be associated with a clinically relevant fusion event. The sample of interest is exposed to both Cas9/gRNA complexes or cocktail of complexes and subsequently analyzed by a negative enrichment assay.

Thus the invention provides methods for the detection of clinically actionable information about a subject. Methods of the invention may be used to with tumor DNA to monitor cancer remission, or to inform immunotherapy treatment. Methods may be used with fetal DNA to detect, for example, mutations characteristic of inherited genetic disorders. Methods may be used to detect and describe mutations and/or alterations in circulating tumor DNA in a blood or plasma sample that also contains an abundance of “normal”, somatic DNA, Methods may be used for directly detecting structural alterations such as translocations, inversions, copy number variations, loss of heterozygosity, or large indels. The subject DNA may include circulating tumor DNA in a patient's blood or plasma, or fetal DNA in maternal blood or plasma.

In certain aspects, the invention provides a method for detecting a structural genomic alteration. The method includes protecting a segment of nucleic acid in a sample by introducing Cas endonuclease/guide RNA complexes that bind to targets that flank a boundary of a genomic alteration, digesting unprotected nucleic acid, and detecting the segment, thereby confirming the presence of the genomic alteration. The digesting step may include exposing the unprotected nucleic acid to one or more exonucleases. Preferably, the Cas endonuclease/guide RNA complexes include guide RNAs with targeting regions complementary to targets that do not appear on the same chromosome in a healthy human genome.

After digestion, the protected segment of nucleic acid may be detected or analyzed by any suitable method. For example, the segment may be detected or analyzed by DNA staining, spectrophotometry, sequencing, fluorescent probe hybridization, fluorescence resonance energy transfer, optical microscopy, electron microscopy, others, or combinations thereof. The segment may be of any suitable length. Methods of the invention are useful for isolation of long fragments of DNA, and the digesting step may include isolating the segment as an intact fragment of DNA with a length of at least five thousand bases. Short fragments may be isolated in some embodiments, e.g., fragments with about 50 to a few hundred bases in length.

The method may include providing a report describing the presence of the genomic alteration in a genome of a subject.

In some embodiments, the sample includes plasma from the subject and the segment is cell-free DNA (cfDNA). The plasma may be maternal plasma and the segment may be of fetal DNA. In certain embodiments, the sample includes plasma from the subject and the segment is circulating tumor DNA (ctDNA). In some embodiments, the sample includes at least one circulating tumor cell from a tumor and the segment is tumor DNA from the tumor cell.

Aspects of the invention provide a method for detecting a mutation. The method includes protecting a segment of a nucleic acid in a sample by introducing first Cas endonuclease/guide RNA complex that binds to a mutation in the nucleic acid and a second such complex that also binds to the same nucleic acid. The first and second Cas endonuclease/guide RNA complexes bind to the nucleic acid to define and protect a segment of the nucleic acid, and—by virtue of the mutation-specific binding of at least the first complex—only bind to, and protect, the segment in the presence of the mutation. The method includes digesting unprotected nucleic acid and detecting the segment, there confirming the presence of the mutation. The digesting step may include exposing the unprotected nucleic acid to one or more exonucleases.

In preferred embodiments, the first Cas endonuclease/guide RNA complex includes a guide RNA with targeting region that binds to the mutation but that does not bind to other variants at a loci of the mutation. The detecting step may include DNA staining, spectrophotometry, sequencing, fluorescent probe hybridization, fluorescence resonance energy transfer, optical microscopy, electron microscopy, others, or combinations thereof. The digesting step may include isolating the segment as an intact fragment of DNA, which fragment may have any suitable length (e.g., about ten to a few hundred bases, a few hundred to a few thousand bases, at least about five thousand bases, etc.). The method may include providing a report describing the presence of the mutation in a genome of a subject.

In some embodiments, the sample includes plasma from the subject and the segment is cell-free DNA (cfDNA). For example, the plasma may be maternal plasma and the segment may be of fetal DNA. In certain embodiments, the sample includes plasma from the subject and the segment is circulating tumor DNA (ctDNA). Optionally, the sample includes at least one circulating tumor cell from a tumor and the segment comprises tumor DNA from the tumor cell.

### BRIEF DESCRIPTION OF THE DRAWINGS

FIG. 1 diagrams a method of detecting a nucleic acid.

FIG. 2 illustrates a method according to an embodiment of the invention.

FIG. 3 diagrams a method for detecting a structural genomic alteration.

FIG. 4 illustrates a sample that includes DNA from a subject.

FIG. 5 shows binding proteins protecting a DNA segment that includes a breakpoint.

FIG. 6 shows the detection of an isolated segment of nucleic acid.

FIG. 7 shows a report describing a structural alteration in nucleic acid from a subject.

FIG. 8 diagrams a method for detecting a mutation.

FIG. 9 illustrates an allele-specific guide RNA for mutation detection.

FIG. 10 illustrates a negative enrichment.

FIG. 11 shows a kit of the invention.

### DETAILED DESCRIPTION

The invention provides methods of detecting nucleic acids within a sample by performing sequential enrichment steps, specifically, a negative enrichment and a positive enrichment. By performing two enrichment, the methods allow detection of nucleic acids present at low abundance in a sample.

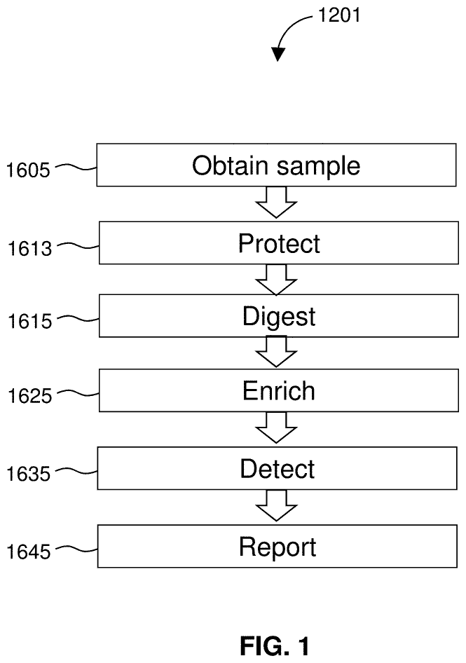

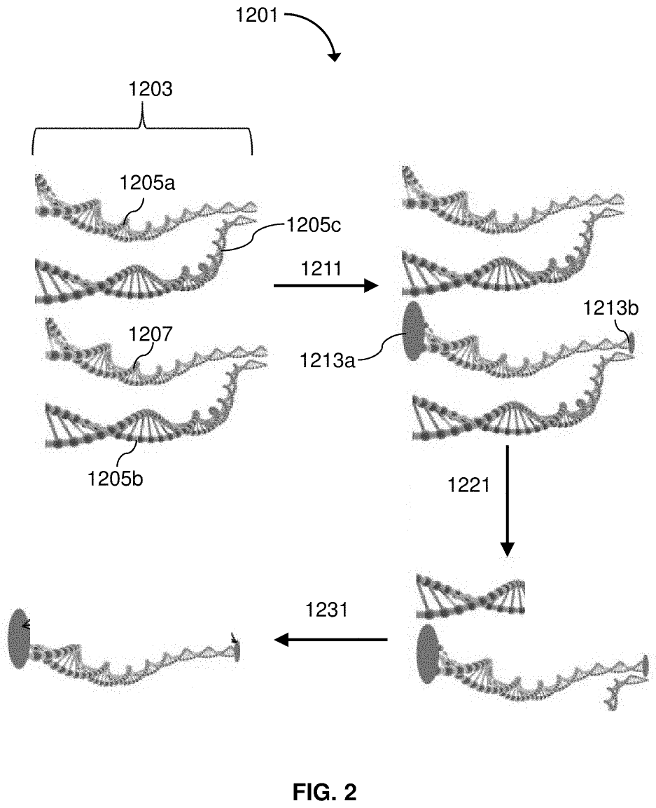

FIG. 1 diagrams a method 1201 of detecting a nucleic acid. The method 1201 may include obtaining 1605 a nucleic acid sample. The method includes protecting 1613 a segment in the sample by binding proteins to ends of the segment. The method 1201 further includes a negative enrichment step of digesting 1615 unprotected nucleic acids followed by a positive enrichment step of enriching 1625 the sample for the protected segment. The method 1201 then includes detecting 1635 the protected segment. The method 1201 may include reporting 1645 that the segment is present in the sample. FIG. 2 illustrates the method 1201. A sample 1203 of nucleic acids 1205a, 1205b, and 1205c including a segment 1207 containing a feature of interest, is provided. The segment 1207 is protected 1211 by allowing proteins 1213a, 1213b to bind to sequences at the ends of the segment 1207. The segment 1207 may be a portion of larger nucleic acid molecule, and the ends of the segment 1207 may not be the ends of a nucleic acid molecule, i.e., the ends may not be free 5′ phosphate groups or free 3′ OH groups. Binding of the proteins to the ends of the segment provides protection against exonuclease digestion. Nucleic acids 1205a, 1205b, and 1205c in the sample 1203 are then digested 1221 by for, example, an exonuclease, but the segment 1207 is protected from digestion. Nucleic acid 1205a is completely degraded, but residual fragments of nucleic acids 1205b and 1205c remain. The sample 1203 is then enriched 1231 for the segment 1207, which removes or minimizes the amount of nucleic acids 1205b and 1205c. The segment 1207 may then be detected by any suitable means.

The positive enrichment allows the segment to be separated from other nucleic acids that are not removed by the digestion step. For example, some nucleic acids may not be fully degraded during the digestion, so they may interfere with detection of the segment. Many methods of purification or enrichment, and any suitable method may be used.

One method for positive enrichment of protein-bound nucleic acids is immunomagnetic separation. Magnetic or paramagnetic particles are coated with an antibody that binds the protein bound to the segment, and a magnetic field is applied to separate particle-bound segment from other nucleic acids. Methods of immunomagnetic purification of biological materials such as cells and macromolecules are known in the art and described in, for example, U.S. Pat. No. 8,318,445; Safarik and Safarikova, Magnetic techniques for the isolation and purification of proteins and peptides, Biomagn Res Technol. 2004; 2:7, doi: 10.1186/1477-044X-2-7, the contents of each of which are incorporated herein by reference. The antibody may be a full-length antibody, a fragment of an antibody, a naturally occurring antibody, a synthetic antibody, an engineered antibody, or a fragment of the aforementioned antibodies. Alternatively or additionally, the particles may be coated with another protein-binding moiety, such as an aptamer, peptide, receptor, ligand, or the like.

Chromatographic methods may be used for positive enrichment. In such methods, the sample is applied to a column, and the segment is separated from other nucleic acids based on a difference in the properties of the segment and the other nucleic acids. Size exclusion chromatography is useful for separating molecules based on differences in size and thus is useful when the segment is larger than the residual nucleic acids left from the digestion step. Methods of size exclusion chromatography are known in the art and described in, for example, Ballou, David P.; Benore, Marilee; Ninfa, Alexander J. (2008). Fundamental laboratory approaches for biochemistry and biotechnology (2nd ed.). Hoboken, N.J.: Wiley. p. 129. ISBN 9780470087664; Striegel, A. M.; and Kirkland, J. J.; Yau, W. W.; Bly, D. D.; Modern Size Exclusion Chromatography, Practice of Gel Permeation and Gel Filtration Chromatography, 2nd ed.; Wiley: NY, 2009, the contents of each of which are incorporated herein by reference.

Ion exchange chromatography uses an ion exchange mechanism to separate analytes based on their respective charges. Thus, ion exchange chromatography can be used with the proteins bound to the segment impart a differential charge as compared to other nucleic acids. Methods of ion exchange chromatography are known in the art and described in, for example,

Small, Hamish (1989). Ion chromatography. New York: Plenum Press. ISBN 0-306-43290-0;

Tatjana Weiss, and Joachim Weiss (2005). Handbook of Ion Chromatography. Weinheim: Wiley-VCH. ISBN 3-527-28701-9; Gjerde, Douglas T.; Fritz, James S. (2000). Ion Chromatography. Weinheim: Wiley-VCH. ISBN 3-527-29914-9; and Jackson, Peter; Haddad, Paul R. (1990). Ion chromatography: principles and applications. Amsterdam: Elsevier. ISBN 0-444-88232-4, the contents of each of which are incorporated herein by reference.

Adsorption chromatography relies on difference in the ability of molecule to adsorb to a solid phase material. Larger nucleic acid molecules are more adsorbent on stationary phase surfaces than smaller nucleic acid molecules, so adsorption chromatography is useful when the segment is larger than the residual nucleic acids left from the digestion step. Methods of adsorption chromatography are known in the art and described in, for example, Cady, 2003, Nucleic acid purification using microfabricated silicon structures. Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; and Wolfe, 2002, Toward a microchip-based solid-phase extraction method for isolation of nucleic acids, Electrophoresis 23:727-733, each incorporated by reference.

Another method for positive enrichment is gel electrophoresis. Gel electrophoresis allows separation of molecules based on differences in their sizes and is thus useful when the segment is larger than the residual nucleic acids left from the digestion step. Methods of gel electrophoresis are known in the art and described in, for example, Tom Maniatis; E. F. Fritsch; Joseph Sambrook. “Chapter 5, protocol 1”. Molecular Cloning—A Laboratory Manual. 1 (3rd ed.). p. 5.2-5.3. ISBN 978-0879691363; and Ninfa, Alexander J.; Ballou, David P.; Benore, Marilee (2009). fundamental laboratory approaches for biochemistry and biotechnology. Hoboken, N.J.: Wiley. p. 161. ISBN 0470087668, the contents of which are incorporated herein by reference.

The proteins that bind to ends of the segment may be any proteins that bind a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference. The protein may be a catalytically inactive form of a nuclease, such as a programmable nuclease described above. The protein may be a transcription activator-like effector (TALE). The protein may be complexed with a nucleic acid that guides the protein to an end of the segment. For example, the protein may be a Cas endonuclease-guide RNA complex.

The unprotected nucleic acid may be digested by any suitable means. Preferably, the unprotected nucleic acid is digested by one or more exonucleases.

The segment may be detected by any means known in the art. For example and without limitation, the segment may be detected by DNA staining, spectrophotometry, sequencing, fluorescent probe hybridization, fluorescence resonance energy transfer, optical microscopy, or electron microscopy. Methods of DNA sequencing are known in the art and described in, for example, Peterson, 2009, Generations of sequencing technologies, Genomics 93(2):105-11; Goodwin, 2016, Coming of age: ten years of next-generation sequencing technologies, Nat Rev Genet 17(6):333-51; and Morey, 2013, A glimpse into past, present, and future DNA sequencing, Mol Genet Metab 110(1-2):3-24, each incorporated by reference. Other methods of DNA detection are known in the art and described in, for example, Xu, 2014, Label-Free DNA Sequence Detection through FRET from a Fluorescent Polymer with Pyrene Excimer to SG, ACS Macro Lett 3(9):845-848, incorporated by reference.

The nucleic acid may be any naturally-occurring or artificial nucleic acid. The nucleic acid may be DNA, RNA, hybrid DNA/RNA, peptide nucleic acid (PNA), morpholino and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The feature of interest may be any feature of a nucleic acid. The feature may be a mutation. For example and without limitation, the feature may be an insertion, deletion, substitution, inversion, amplification, duplication, translocation, copy number variation, or polymorphism. The feature may be a nucleic acid from an infectious agent or pathogen. For example, the nucleic acid sample may be obtained from an organism, and the feature may contain a sequence foreign to the genome of that organism.

The segment may be from a sub-population of nucleic acid within the nucleic acid sample. For example, the segment may contain cell-free DNA, such as cell-free fetal DNA or circulating tumor DNA.

The nucleic acid sample may come from any source. For example, the source may be an organism, such as a human, non-human animal, plant, or other type of organism. The sample may be a tissue sample from an animal, such as blood, serum, plasma, skin, urine, saliva, semen, feces, phlegm, conjunctiva, gastrointestinal tract, respiratory tract, vagina, placenta, uterus, oral cavity or nasal cavity. The sample may be a liquid biopsy.

The nucleic acid sample may come from an environmental source, such as a soil sample or water sample, or a food source, such as a food sample or beverage sample. The sample may comprise nucleic acids that have been isolated, purified, or partially purified from a source. Alternatively, the sample may not have been processed.

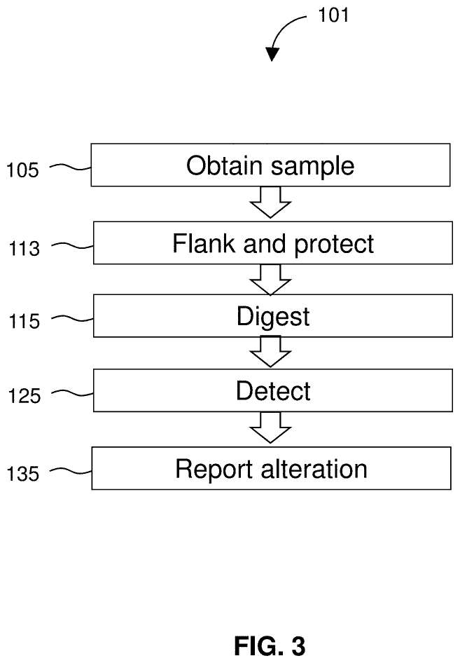

FIG. 3 diagrams a method 101 for detecting a structural genomic alteration. The method 101 includes obtaining a sample that includes DNA from a subject. Binding proteins are introduced to protect 113 a segment of nucleic acid in the sample. The binding proteins bind to specific targets that flank a boundary of a genomic alteration. The method 101 includes digesting 115 unprotected nucleic acid and detecting 125 the segment, there confirming the presence of the genomic alteration in the subject. A report 135 may be provided that describes the alteration as being present in the subject.

Any suitable structural genomic alteration may be detected using the method 101. Suitable structural alterations may include, for example, inversions, translocations, copy number variations, or gene duplications. Binding proteins are used that will flank a boundary of the structural alteration only when the alteration is present. For example, binding proteins may be used that—in the absence of the alteration—bind to different chromosomes of a human genome. Methods of the invention are used to detect the alteration in a DNA sample from a subject.

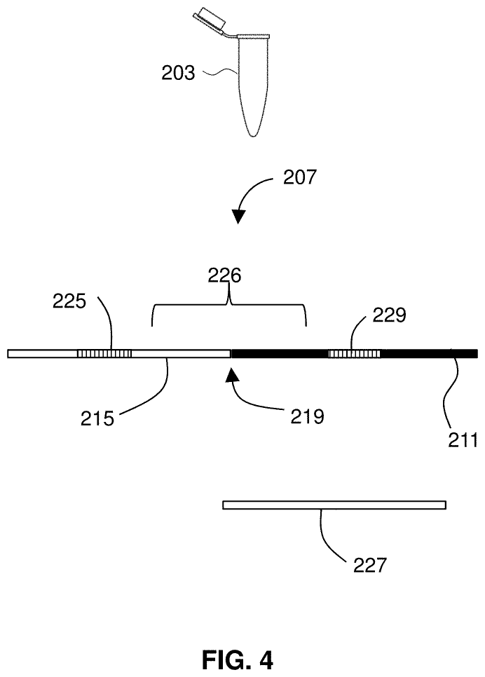

FIG. 4 illustrates a sample 203 that includes DNA 207 from a subject. The DNA 207 may be any suitable DNA and in preferred embodiments includes cell-free DNA, such as circulating tumor DNA (ctDNA) or fetal DNA from maternal blood or plasma. The sample may include plasma from the subject in which the segment is cell-free DNA (cfDNA). In some embodiments, the sample 203 includes maternal plasma and fetal DNA. In certain embodiments, ctDNA is in the sample 203. In some embodiments, the sample 203 includes at least one circulating tumor cell from a tumor and the segment comprises tumor DNA from the tumor cell.

Methods may include detection or isolation of circulating tumour cells (CTCs) from a blood sample. Cytometric approaches use immunostaining profiles to identify CTCs. CTC methods may employ an enrichment step to optimize the probability of rare cell detection, achievable through immune-magnetic separation, centrifugation or filtration. Cytometric CTC technology includes the CTC analysis platform sold under the trademark CELLSEARCH by Veridex LLC (Huntingdon Valley, Pa.). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

In the illustrated example, the DNA 207 has a portion 211 that originated from a first chromosome and a second portion 215 that originated from a different chromosome. By virtue of a translocation between the two chromosomes, the DNA 207 includes a breakpoint 219 of the translocation. The DNA also includes a first binding target 229 for a first binding protein and a second binding target 225 for second binding protein. The two binding targets 229, 225 flank the breakpoint 219, which lies in a segment 226 between the two binding targets. The sample may include other nucleic acid 227 that does not include the targets or the breakpoint. The method includes binding the binding proteins to the targets 225, 229.

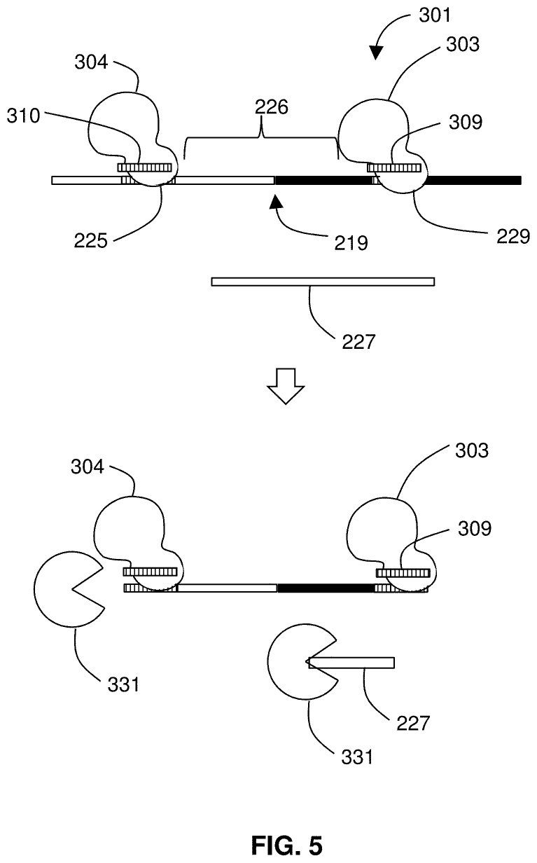

FIG. 5 shows binding proteins 301 being introduced to protect 113 the segment 226 of nucleic acid where the breakpoint 219 lies. The binding proteins 301 bind to specific targets that flank a boundary of a genomic alteration. The depicted method for isolating the segment 226 may be described as a negative enrichment. Contrary to the standard approach of enriching a specific genomic sequence of interest away from a heterogeneous background of DNA by trying to fish out the sequence of interest from the ocean of unwanted sequence, the depicted approach dries up the ocean, leaving behind the target sequence of interest. Methods may be used to perform such an approach to enrich for long DNA fragments (˜50-100 kb) of interest. The fragment may be detected or analyzed, e.g., sequenced by NGS or a long read sequencing platform such as Oxford Nanopore or PacBio. Methods may be used to isolate any length fragment (e.g., 100 bases, 150 bases, 175 bases, etc. . . . ) that includes a boundary of an alteration, such as a breakpoint of a fusion event.

In a population of DNA where a clinically informative fusion event is not present, the genomic DNA is digested down to the size of a DNA sequence equivalent to the amount of sequence protected by a single Cas9/gRNA complex. However, in those samples where a clinically informative fusion is present, both binding proteins 301 will be located on the same DNA strand and therefore protecting the segment 226 between the proteins 301 from DNA degradation.

In a preferred embodiment, the binding proteins 301 are provided by Cas endonuclease/guide RNA complexes. Embodiments of the invention use proteins that are originally encoded by genes that are associated with clustered regularly interspaced short palindromic repeats (CRISPR) in bacterial genomes. Preferred embodiments use a CRISPR-associated (Cas) endonuclease. For such embodiments, the binding protein in a Cas endonuclease complexed with a guide RNA that targets the Cas endonuclease to a specific sequence. Any suitable Cas endonuclease or homolog thereof may be used. A Cas endonuclease may be Cas9 (e.g., spCas9), catalytically inactive Cas (dCas such as dCas9), Cpf1, C2c2, others, modified variants thereof, and similar proteins or macromolecular complexes. A first Cas endonuclease/guide RNA complex includes a first Cas endonuclease 303 and a first guide RNA 309. A second Cas endonuclease/guide RNA complex includes a second Cas endonuclease 304 and a second guide RNA 310.

In the preferred embodiments, the two Cas endonuclease complexes (or sets of complexes if nickases are used) define the locus that includes a junction of a known chimeric/fusion chromosome/gene, i.e., the boundary 219. The complexes protect the segment 226 of nucleic acid that includes the boundary 219. One or more exonuclease 331 is used to digest 115 unprotected nucleic acid. In some embodiments, ExoIII and ExoVII destroy all DNA that does not include both binding/protecting sites. The only DNA that remains includes the junction, or boundary 219, of the known chimera (fusion).

As a result of digestion 115 by exonuclease 331, unprotected nucleic acid 227 is removed from the sample. What remains is the segment 226 containing the breakpoint 219, to which the first Cas endonuclease 303 and second Cas endonuclease 304 may remain bound. The method 101 further includes detecting 125 the segment 226 as present after the digestion step. Any suitable detection technique may be used such as, for example, DNA staining; spectrophotometry; sequencing; fluorescent probe hybridization; fluorescence resonance energy transfer; optical microscopy; or electron microscopy.

The Cas9/gRNA complexes may be subsequently or previously labeled using standard procedures, and single molecule analysis identifying coincidence signal of the two Cas9/gRNA complexes located on the same DNA molecule identifies the presence of the clinically informative fusion of interest. The complexes may be fluorescently labeled, e.g., with distinct fluorescent labels such that detecting involves detecting both labels together (e.g., after a dilution into fluid partitions). The complexes may be labeled with a FRET system such that they fluoresce only when bound to the same segment. Preferred embodiments of analysis does not require PCR amplification and therefore significantly reduces cost and sequence bias associated with PCR amplification. Sample analysis can also be performed by a number of approaches such as NGS etc. However, many analytical platforms may require PCR amplification prior to analysis. Therefore, preferred embodiments of analysis of the reaction products include single molecule analysis that avoid the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

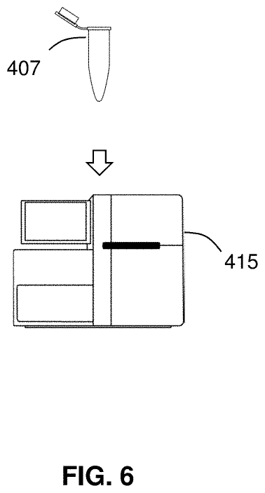

FIG. 6 shows the detection 125 of the isolated segment 226 of the nucleic acid. The digestion 115 provides a reaction product 407 that includes principally only the segment 226 of nucleic acid, as well as any spent reagents, Cas endonuclease complexes, exonuclease, nucleotide monophosphates, or pyrophosphate as may be present. The reaction product 407 may be provided as an aliquot (e.g., in a micro centrifuge tube such as that sold under the trademark EPPENDORF by Eppendorf North America (Hauppauge, N.Y.) or glass cuvette). The reaction product 407 may be disposed on a substrate. For example, the reaction product may be pipetted onto a glass slide and subsequently combed or dried to extend the fragment 226 across the glass slide. The reaction product may optionally be amplified. Optionally, adaptors are ligated to ends of the reaction product, which adaptors may contain primer sites or sequencing adaptors. The presence of the segment 226 in the reaction product 407 may then be detected using an instrument 415.

The fragment 226 may be detected, sequenced, or counted. Where a plurality of fragment 226 are present or expected, the fragment may be quantified, e.g., by qPCR.

In certain embodiments, the instrument 415 is a spectrophotometer, and the detection 125 includes measuring the adsorption of light by the reaction product 407 to detect the presence of the segment 226. The method 101 may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance). By the described methods, a sample can be assayed for a genomic structural alteration using a technique that is inexpensive, quick, and reliable. Methods of the disclosure are conducive to high throughput embodiments, and may be performed, for example, in droplets on a microfluidic device, to rapidly assay a large number of aliquots from a sample for one or any number of genomic structural alterations.

The Cas endonuclease/guide RNA complexes can be designed to flank suspected gene fusions, or may be designed without a priori knowledge of any such alteration, but introduced to sample nucleic acid in pairs that include guide RNAs with targeting regions complementary to targets that do not appear on the same chromosome in a healthy human genome. The complexes bind to healthy DNA on different chromosomes, so detecting a segment via the described method 101 indicates the presence of a structural alteration in the subject's DNA.

When a genomic structural alteration is thus detected, a report may be provided 135 to, for example, describe the alteration in a patient.

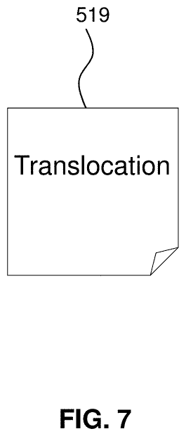

FIG. 7 shows a report 519 as may be provided in certain embodiments. The report preferably includes a description of the structural alteration in the subject (e.g., a patient). The method 101 for detecting structural alterations may be used in conjunction with a method of describing mutations (e.g., as described herein). Either or both detection process may be performed over any number of loci in a patient's genome or preferably in a patient's tumor DNA. As such, the report 519 may include a description of a plurality of structural alterations, mutations, or both in the patient's genome or tumor DNA. As such, the report 519 may give a description of a mutational landscape of a tumor.

Knowledge of a mutational landscape of a tumor may be used to inform treatment decisions, monitor therapy, detect remissions, or combinations thereof. For example, where the report 519 includes a description of a plurality of mutations, the report 519 may also include an estimate of a tumor mutation burden (TMB) for a tumor. It may be found that TMB is predictive of success of immunotherapy in treating a tumor, and thus methods described herein may be used for treating a tumor.

Methods of the invention thus may be used to detect and report clinically actionable information about a patient or a tumor in a patient. For example, the method 101 may be used to provide 135 a report describing the presence of the genomic alteration in a genome of a subject. Additionally, protecting 113 a segment 226 of DNA and digesting 115 unprotected DNA provides a method for isolation or enrichment of DNA fragments, i.e., the protected segment. It may be found that the described enrichment technique is well-suited to the isolation/enrichment of arbitrarily long DNA fragments, e.g., thousands to tens of thousands of bases in length.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genomic regions that would be used to determine TMB for immuno-oncology associated therapeutic treatments. In such applications, the negative enrichment technology is utilized to enrich large regions (>50 kb) within the genome of interest.

In preferred embodiments, the invention provides methods 101 for detecting structural alterations and/or methods for detecting mutations in DNA.

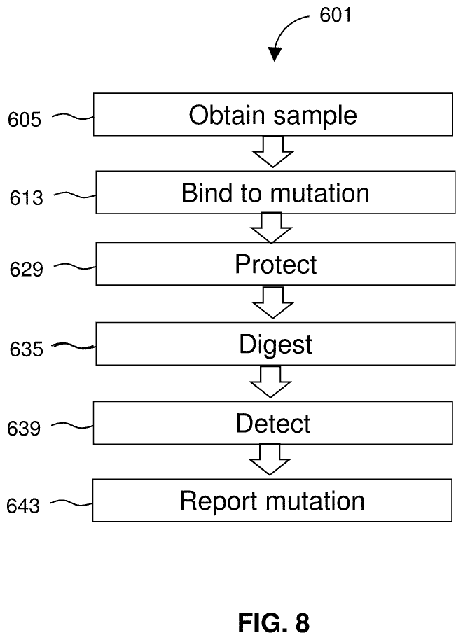

FIG. 8 diagrams a method 601 for detecting a mutation. The method 601 includes obtaining 605 a sample that includes DNA from a subject. The sample is exposed to a first Cas endonuclease/guide RNA complex that binds 613 to a mutation in a sequence-specific fashion. The method 601 includes protecting 629 a segment of nucleic acid in a sample by introducing the first Cas endonuclease/guide RNA complex (that binds to a mutation in the nucleic acid) and a second Cas endonuclease/guide RNA complex that also binds to the nucleic acid. Unprotected nucleic acid is digested 635. For example, one or more exonucleases may be introduced that promiscuously digest unbound, unprotected nucleic acid. While the exonucleases act, the segment containing the mutation of interest is protected by the bound complexes and survives the digestion step 635 intact. The method 601 includes detecting 639 the segment, there confirming the presence of the mutation. A report may be provided 643 that describes the mutation as being present in the subject.

The method 601 uses the idea of mutation-specific gene editing, or “allele-specific” gene editing, which may be implemented via complexes that include a Cas endonuclease and an allele-specific guide RNA.

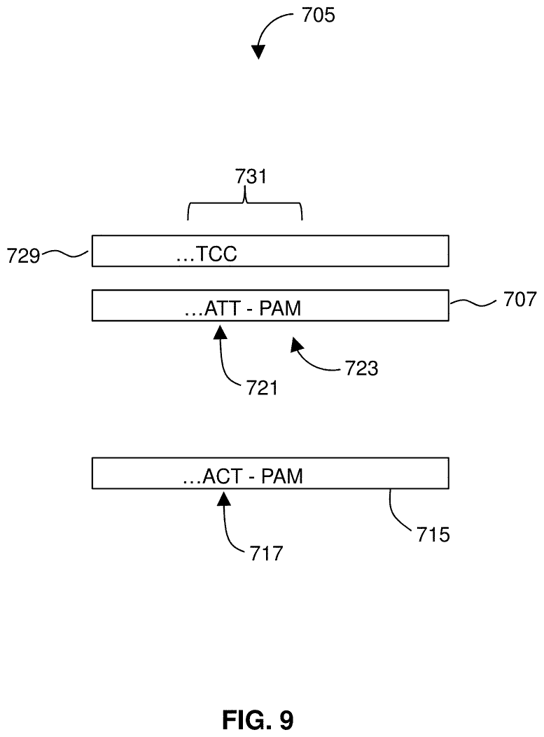

FIG. 9 illustrates the operation of allele-specific guide RNA for mutation detection. A sample 705 may contain a mutant fragment 707 of DNA, a wild-type fragment 715 of DNA, or both. A locus of interest is identified where a mutation 721 may be present proximal to, or within, a protospacer adjacent motif (PAM) 723. When the wild-type fragment 715 is present, it may contain a wild-type allele 717 at a homologous location in the fragment 715, also proximal to, or within, a PAM. A guide RNA 729 is introduced to the sample that has a targeting portion 731 complementary to the portion of the mutant fragment 707 that includes the mutation 721. When a Cas endonuclease is introduced, it will form a complex with the guide RNA 729 and bind to the mutant fragment 707 but not to the wild-type fragment 715. The first Cas endonuclease/guide RNA complex includes a guide RNAs with targeting region that binds to the mutation but that does not bind to other variants at a loci of the mutation.

The described methodology may be used to target a mutation 721 that is proximal to a PAM 723, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM include NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over a genome at such frequency that the potentially detectable mutations are abundant enough as to be representative of mutations over the genome at large. In such cases, allele-specific negative enrichment may be used to detect mutations in targetable portions of a genome. Moreover, the method 601 may be used to determine a number of mutations over the representative, targetable portion of the genome. Since the targetable portion of the genome is representative of the genome overall, the number of mutations may be used to infer a mutational burden for the genome overall. Where the sample includes tumor DNA and the mutations are detected in tumor DNA, the method 601 may be used to give a tumor mutation burden.

The method 601 includes the described negative enrichment, in which a segment of nucleic acid in a sample is protected 629 by a first Cas endonuclease/guide RNA complex (that binds to a mutation in the nucleic acid) and a second Cas endonuclease/guide RNA complex that also binds to the nucleic acid.

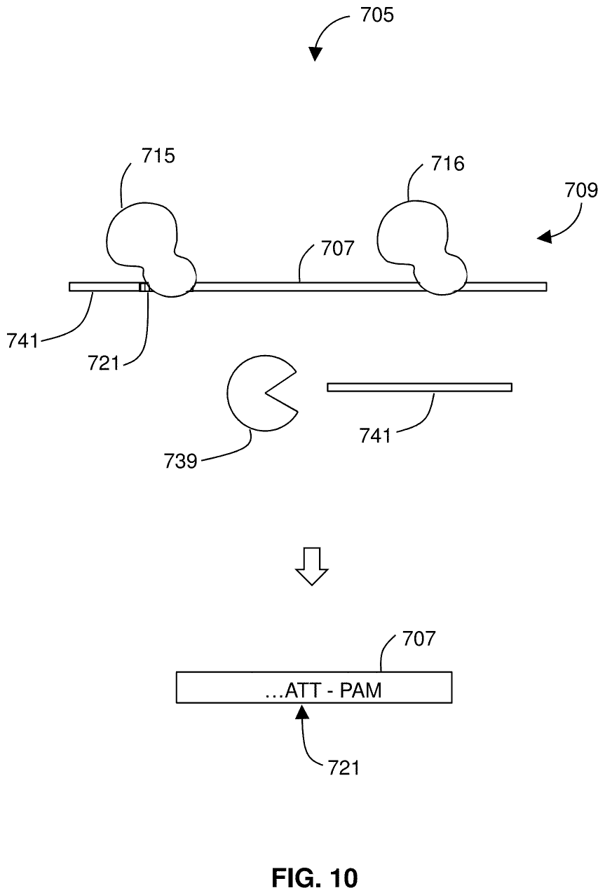

FIG. 10 illustrates operation of the negative enrichment. The sample 705 includes DNA 709 from a subject. The sample 705 is exposed to a first Cas endonuclease/guide RNA complex 715 that binds to a mutant fragment 707 mutation in a sequence-specific fashion. Specifically, the complex 715 binds to the mutation 721 in a sequence-specific manner. A segment of the nucleic acid 709, i.e., the mutant fragment 707, is protected by introducing the first Cas endonuclease/guide RNA complex 715 (that binds to a mutation in the nucleic acid) and a second Cas endonuclease/guide RNA complex 716 that also binds to the nucleic acid. Unprotected nucleic acid 741 is digested. For example, one or more exonucleases 739 may be introduced that promiscuously digest unbound, unprotected nucleic acid 741. While the exonucleases 739 act, the segment containing the mutation of interest, the mutant fragment 707, is protected by the bound complexes 715, 716 and survives the digestion step intact.

The described steps including the digestion by the exonuclease 739 leaves a reaction product that includes principally only the mutant segment 707 of nucleic acid, as well as any spent reagents, Cas endonuclease complexes, exonuclease 739, nucleotide monophosphates, and pyrophosphate as may be present. The method 601 includes detecting 639 the segment 707 (which includes the mutation 721). Any suitable technique may be used to detect 639 the segment 707. For example, detection may be performed using DNA staining, spectrophotometry, sequencing, fluorescent probe hybridization, fluorescence resonance energy transfer, optical microscopy, electron microscopy, others, or combinations thereof. Detecting the mutant segment 707 indicates the presence of the mutation in the subject (i.e., a patient), and the a report may be provided describing the mutation in the patient.

A feature of the method 601 is that a specific mutation may be detected by a technique that includes detecting only the presence or absence of a fragment of DNA, and it need not be necessary to sequence DNA from a subject to describe mutations. The method 601, the method 101, or both may be performed in fluid partitions, such as in droplets on a microfluidic device, such that each detection step is binary (or “digital”). For example, droplets may pass a light source and photodetector on a microfluidic chip and light may be used to detect the presence of a segment of DNA in each droplet (which segment may or may not be amplified as suited to the particular application circumstance).

The method 601 uses a double-protection to select one or both ends of DNA segments. The gRNA selects for a known mutation on one end. If it doesn't find the mutation, no protection is provided and the molecule gets digested. The remaining molecules are either counted or sequenced. The method 601 is well suited for the analysis of small portions of DNA, degraded samples, samples in which the target of interest is extremely rare, and particularly for the analysis of maternal serum (e.g., for fetal DNA) or a liquid biopsy (e.g., for ctDNA).

The method 601 and the method 101 include a negative enrichment step that leaves the target loci of interest intact and isolated as a segment of DNA. The methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. Embodiments of the invention provide kits that may be used in performing methods described herein.

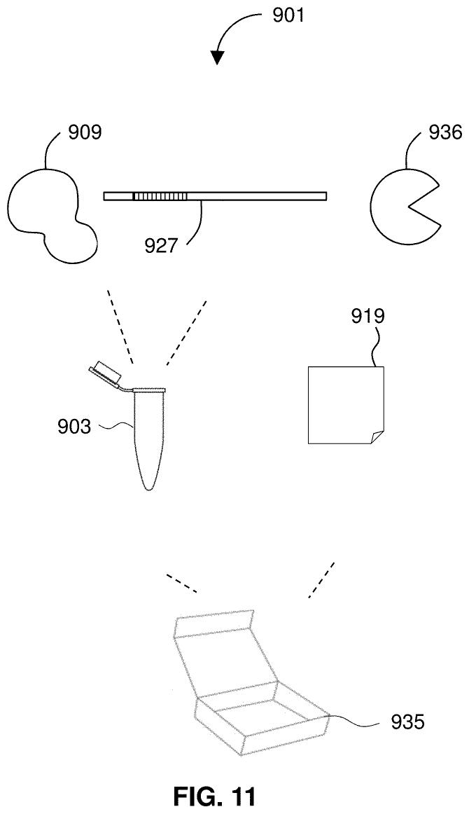

FIG. 11 shows a kit 901 of the invention. The kit 901 may include reagents 903 for performing the steps described herein. For example, the reagents 903 may include one or more of a Cas endonuclease 909, a guide RNA 927, and exonuclease 936. The kit 901 may also include instructions 919 or other materials such as pre-formatted report shells that receive information from the methods to provide a report (e.g., by uploading from a computer in a clinical services lab to a server to be accessed by a geneticist in a clinic to use in patient counseling). The reagents 903, instructions 919, and any other useful materials may be packaged in a suitable container 935. Kits of the invention may be made to order. For example, an investigator may use, e.g., an online tool to design guide RNA and reagents for the performance of methods 101, 601. The guide RNAs 927 may be synthesized using a suitable synthesis instrument. The synthesis instrument may be used to synthesize oligonucleotides such as gRNAs or single-guide RNAs (sgRNAs). Any suitable instrument or chemistry may be used to synthesize a gRNA. In some embodiments, the synthesis instrument is the MerMade 4 DNA/RNA synthesizer from Bioautomation (Irving, Tex.). Such an instrument can synthesize up to 12 different oligonucleotides simultaneously using either 50, 200, or 1,000 nanomole prepacked columns. The synthesis instrument can prepare a large number of guide RNAs 927 per run. These molecules (e.g., oligos) can be made using individual prepacked columns (e.g., arrayed in groups of 96) or well-plates. The resultant reagents 903 (e.g., guide RNAs 917, endonuclease(s) 909, exonucleases 936) can be packaged in a container 935 for shipping as a kit.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for detecting nucleic acid in a sample, the method comprising:
protecting a nucleic acid of interest in a sample by binding proteins to ends of the nucleic acid;
digesting unprotected nucleic acid;
enriching the sample for the nucleic acid; and
detecting the nucleic acid.

2. The method of claim 1, wherein the proteins each comprise a Cas endonuclease complexed with a guide RNA that targets the Cas endonuclease to an end of the nucleic acid.

3. The method of claim 2, wherein digesting the unprotected nucleic acid includes introducing an exonuclease into the sample.

4. The method of claim 1, wherein the enriching step comprises connecting the protected nucleic acid to a particle or column and removing other components of the sample.

5. The method of claim 4, wherein the particle comprises an agent that binds to at least one of the proteins.

6. The method of claim 4, wherein the particle comprises magnetic or paramagnetic material, and wherein the enriching step comprises applying a magnetic field to separate the particle-bound protected segment from the other components.

7. The method of claim 1, wherein the enriching step comprises applying the sample to a column.

8. The method of claim 7, wherein the protected segment is separated from unprotected nucleic acid by size exclusion, ion exchange, or adsorption.

9. The method of claim 1, wherein the enriching step comprises gel electrophoresis.

10. The method of claim 2, wherein the Cas endonucleases are enzymatically inactive.

11. The method of claim 1, wherein the digesting step comprises exposing the unprotected nucleic acid to one or more exonucleases.

12. The method of claim 1, wherein the detecting step includes one selected from the group consisting of DNA staining, spectrophotometry, sequencing, fluorescent probe hybridization, fluorescence resonance energy transfer, optical microscopy, and electron microscopy.

13. The method of claim 1, wherein detecting the nucleic acid includes identifying a mutation in the nucleic acid.

14. The method of claim 13, wherein identifying the mutation includes one selected from the group consisting of: sequencing the nucleic acid, allele-specific amplification, and hybridizing a probe the nucleic acid.

15. The method of claim 1, wherein the sample comprising blood or plasma, and the nucleic acid comprises DNA from a tumor.

16. The method of claim 1, wherein the nucleic acid sample comprises a liquid biopsy.

17. The method of claim 16, wherein the nucleic acid comprises circulating tumor DNA.

18. The method of claim 1, wherein the sample comprises maternal plasma, and wherein the nucleic acid comprises fetal DNA.

19. A method for detecting a mutation in a nucleic acid sample, the method comprising:
protecting, in a nucleic acid sample, a segment that includes a mutation by binding a first protein to the mutation and a second protein to the segment;
digesting unprotected nucleic acid;
enriching the sample for the segment; and
detecting the segment.

20. The method of claim 19, wherein at least one of the first protein and the second protein is a Cas endonuclease.
